(19) United States
(12) Patent Application Publication
(10) Pub. No.: US 2024/0374217 A1
(43) Pub. Date: Nov. 14, 2024

(54) SYSTEMS AND METHODS FOR CHARACTERIZING BRAIN ACTIVITY

(71) Applicant: The Texas A&M University System, College Station, TX (US)

(72) Inventors: James E. Hubbard, Jr., College Station, TX (US); Mark J. Balas, College Station, TX (US); Tristan D. Griffith, Bryan, TX (US)

(73) Assignee: The Texas A&M University System, College Station, TX (US)

(21) Appl. No.: 18/035,105

(22) PCT Filed: Dec. 2, 2021
(86) PCT No.: PCT/US2021/061566

§ 371 Date: May 2, 2023

Related U.S. Application Data
(60) Provisional application No. 63/120,244, filed on Dec. 2, 2020.

(51) Int. Cl. A61B 5/00; A61B 5/117; A61B 5/291; A61B 5/31; A61B 5/372

(52) U.S. Cl. CPC A61B 5/7239; A61B 5/117; A61B 5/291; A61B 5/31; A61B 5/372; A61B 5/6814; A61B 5/7246; A61B 5/7257; A61B 5/7264; A61B 5/7278; A61B 5/742

(57) ABSTRACT

A method of characterizing brain activity includes receiving an electroencephalogram (EEG) output. In addition, the method includes determining a mathematical model of a brain using the EEG output, wherein the mathematical model comprises a plurality of ordinary differential equations (ODEs) that are determined based on the EEG output. Further, the method includes characterizing brain activity of a subject using the mathematical model.

### CROSS-REFERENCE TO RELATED APPLICATIONS

This application claims benefit of U.S. provisional patent application Ser. No. 63/120,244 filed Dec. 2, 2020, and entitled “Method of Subject Identification Using Brain Wave Patterns,” which is hereby incorporated herein by reference in its entirety.

### STATEMENT REGARDING FEDERALLY SPONSORED RESEARCH OR DEVELOPMENT

Not applicable.

### BACKGROUND

There has been a strong desire to better understand the inner workings of the human brain. Various tests and methods have been developed for detecting or measuring brain activity. The goal of these tests is to gain better insight into how the human brain performs cognitive thought and functional control of the human body.

### SUMMARY

Some embodiments disclosed herein are directed to a method of characterizing brain activity. In an embodiment, the method includes receiving an electroencephalogram (EEG) output. In addition, the method includes determining a mathematical model of a brain using the EEG output, wherein the mathematical model comprises a plurality of ordinary differential equations (ODEs) that are determined based on the EEG output. Further, the method includes characterizing brain activity of a subject using the mathematical model.

Some embodiments disclosed herein are directed to a non-transitory, machine-readable medium, storing instructions, which, when executed by a processor of an electronic device, cause the processor to: receive an electroencephalogram (EEG) output; determine a mathematical model of a brain using the EEG output, wherein the mathematical model comprises a plurality of ordinary differential equations (ODEs) that are determined based on the EEG output; and characterize brain activity of a subject using the mathematical model.

Some embodiments disclosed herein are directed to a system including a plurality of electrodes that are configured to detect electrical impulses within a brain of a subject. In addition, the system includes an electronic device coupled to the plurality of electrodes. The electronic device is configured to: receive an electroencephalogram (EEG) output; determine a mathematical model of a brain using the EEG output, wherein the mathematical model comprises a plurality of ordinary differential equations (ODEs) that are determined based on the EEG output; receive additional EEG output from the subject from the plurality of electrodes; update the mathematical model using the additional EEG output; and characterize brain activity of the subject using the mathematical model.

Embodiments described herein comprise a combination of features and characteristics intended to address various shortcomings associated with certain prior devices, systems, and methods. The foregoing has outlined rather broadly the features and technical characteristics of the disclosed embodiments in order that the detailed description that follows may be better understood. The various characteristics and features described above, as well as others, will be readily apparent to those skilled in the art upon reading the following detailed description, and by referring to the accompanying drawings. It should be appreciated that the conception and the specific embodiments disclosed may be readily utilized as a basis for modifying or designing other structures for carrying out the same purposes as the disclosed embodiments. It should also be realized that such equivalent constructions do not depart from the spirit and scope of the principles disclosed herein.

### BRIEF DESCRIPTION OF THE DRAWINGS

For a detailed description of various exemplary embodiments, reference will now be made to the accompanying drawings in which:

FIG. 1 is a schematic diagram of a system for characterizing the brain activity of a subject according to some embodiments; and

FIG. 2 is a block diagram of a method for characterizing the brain activity of a subject according to some embodiments.

### DETAILED DESCRIPTION

As previously described, various tests and methods have been developed for detecting or measuring brain activity with a goal of better of understanding the inner workings of the human brain. For instance, functional magnetic resonance imaging (fMRI) is a scanning method whereby brain activity is measured by detecting blood flow changes in the brain. Through an fMRI process, brain activity can be imaged to provide insights to researchers. However, fMRI requires expensive scanning equipment and may not be suitable for measuring or detecting brain activity in all circumstances (e.g., such as when a subject is performing a task).

Alternatively, researchers have attempted to glean insight of brain activity via electroencephalograms (EEGs). An EEG involves coupling multiple electrodes to the external surface of a subject's head to measure the electrical activity within the brain. The cells of the brain communicate via electrical impulses, and thus the aim of an EEG is to detect these electrical impulses to measure the subject's brain activity. However, the measured signals from an EEG have a significant amount of noise. In some cases, noise may contribute to approximately half of the captured electrical signals during an EEG. Thus, there is a need for improved techniques for processing and analyzing EEG data so as to allow for more useful insights of human brain function.

Accordingly, embodiments disclosed herein include methods and associated systems for characterizing the brain activity of a subject via output obtained from an EEG. In some embodiments, the systems and methods disclosed herein may generate a mathematical model(s) for approximating the function of the subject's brain. These models may allow isolation of the underlying linear and non-linear patterns associated with the EEG output signals, which may then be used to make useful characterizations of the brain activity of the subject. For instance, in some embodiments, the systems and methods herein may be used to identify the particular individual who contributed the EEG output signals by analyzing features of the model(s) that are specific and unique to each individual person. As another example, in some embodiments, the systems and methods disclosed herein may be used to determine the cognitive state of the subject during the EEG test via analysis of feature(s) of the models. Thus, through use of the systems and methods disclosed herein, further useful insights may be obtained from an EEG which may facilitate further understanding of human thought and brain function. The systems and method herein may be useful on their own or may be utilized as a supplement to other brain analysis techniques, such as fMRI.

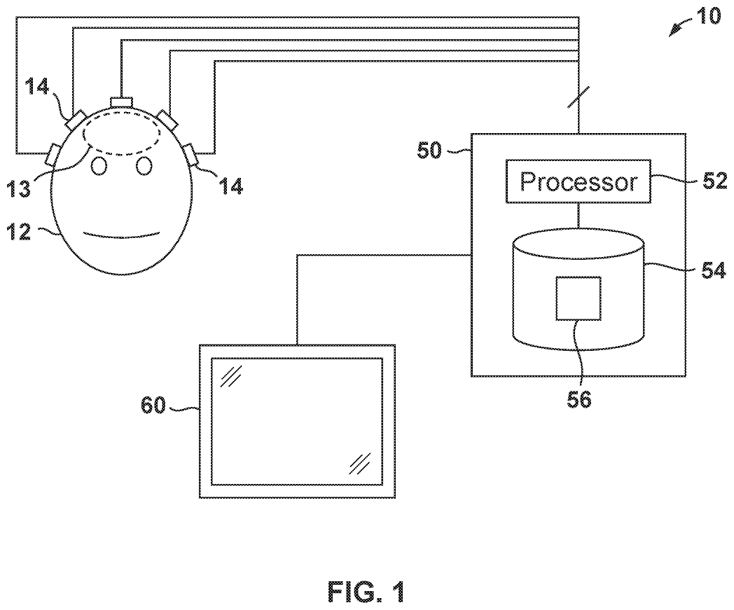

Referring now to FIG. 1, a system 10 for characterizing the brain activity of a subject 12 is shown. The system 10 comprises a plurality of electrodes 14 coupled to the head of the subject 12 and an electronic device 50 coupled to the electrodes 14.

The electrodes 14 may comprise electrodes that are used for an EEG. Thus, the information collected by the electrodes 14 may comprise data that is received during an EEG test, which may comprise signals that are indicative of the electrical impulses generated within the brain 13 of subject 12. The electrodes 14 may be coupled to the external surface of the head of subject 12 via any suitable manner. For instance, in some embodiments, the electrodes 14 are directly attached (e.g., via tape, adhesive, suction cup, etc.) to the scalp of the subject 12. In some examples, the electrodes are incorporated within a cap, visor, helmet, head band, or other object that may be placed on the head of subject 12. In addition, the electrodes 14 may be arranged on the head of subject 12 in any suitable pattern or arrangement. For instance, in some embodiments the electrodes 14 are arranged in the internationally recognized 10-10 or 10-20 systems on the head of subject 12.

The electronic device 50 is coupled to the plurality of electrodes 14 such that signals that are output by the electrodes 14 may be communicated to the electronic device 50 during operations. The electronic device 50 may comprise any suitable device (or collection of devices) that may receive the output from electrodes and execute machine-readable instructions. For instance, in some embodiments, electronic device 50 comprises a computer (e.g., desktop computer, laptop computer, tablet computer), server, smartphone, etc. or a collection of such devices.

The electronic device 50 may comprise a processor 52 and memory 54. The processor 52 may comprise any suitable processing device, such as a microcontroller, central processing unit (CPU), graphics processing unit (GPU), timing controller (TCON), scaler unit, or a combination thereof. The processor 52 executes machine-readable instructions (e.g., machine-readable instructions 56) stored on memory 54, thereby causing the processor 52 to perform some or all of the actions attributed herein to the electronic device 50. In general, processor 52 fetches, decodes, and executes instructions (e.g., machine-readable instructions 56). In addition, processor 52 may also perform other actions, such as, making determinations, detecting conditions or values, etc., and communicating signals. If processor 52 assists another component in performing a function, then processor 52 may be said to cause the component to perform the function.

The memory 54 may comprise volatile storage (e.g., random access memory (RAM)), non-volatile storage (e.g., read-only memory (ROM), flash storage, etc.), or combinations of both volatile and non-volatile storage. Data read or written by the processor 52 when executing machine-readable instructions 56 can also be stored on memory 54. Memory 54 may comprise “non-transitory machine-readable medium.”

The processor 52 may comprise one processing device or a plurality of processing devices that are distributed within electronic device 50 (or across a plurality of such electronic devices 50). Likewise, the memory 54 may comprise one memory device or a plurality of memory devices that are distributed within the electronic device 50 (or a plurality of such electronic devices 50).

During operations, the electronic device 50 may receive the output of the electrodes 14. In some embodiments, the output from the electrodes 14 may be directly provided to the electronic device via a wired or wireless connection. In addition, in some embodiments, the output from the electrodes 14 may be received by a separate device or system, and then may be delivered (e.g., again via a wired and/or wireless connection) to the electronic device 50. In some embodiments, the output from the electrodes 14 may be collected and stored in a memory (e.g., which may be similar to memory 54), and subsequently, the output from electrodes 14 may be provided to electronic device 50.

Once the output from electrodes 14 is received by electronic device 50, processor 52 may execute machine-readable instructions 56 stored on memory 54 to analyze the detected electrical impulses from the brain 13 of subject 12 and draw useful conclusions regarding the subject 12 and/or the brain activity (e.g., including the cognitive state) of the subject 12. In some embodiments, the electronic device 50 may generate an output that is indicative of the conclusions or associated analysis that is provided to an output device 60 coupled to (or integrated with) electronic device 50. For instance, in some embodiments, the output device 60 comprises an electronic display panel (e.g., liquid crystal display (LCD), light emitting diode display (LED display) such as an organic LED display, micro-LED display, etc.), and the electronic device 50 may generate an output comprising graphics, text, and/or numeric elements that is/are displayed on the electronic display panel of output device 60 so that a user (e.g., researcher, physician, etc.) may view the output.

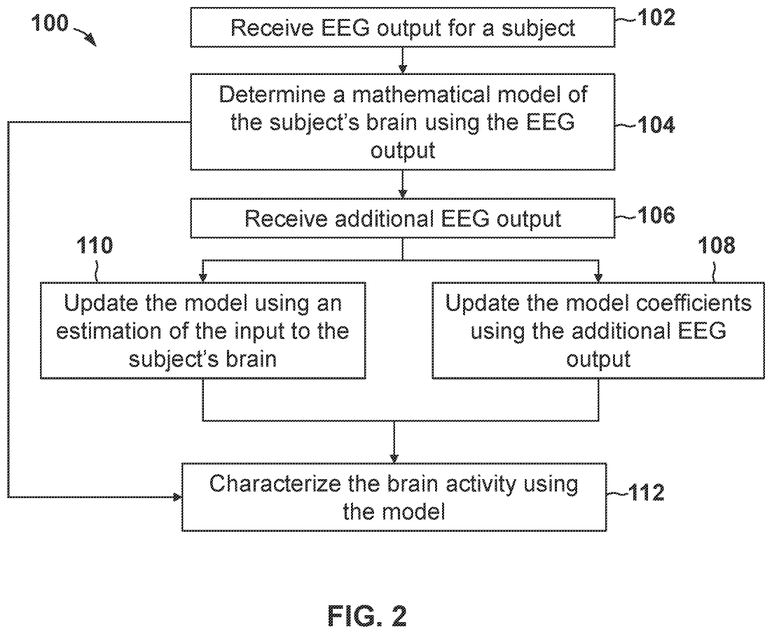

Referring now to FIG. 2, a method 100 of characterizing brain activity using EEG output according to some embodiments is shown. The method 100 may be performed wholly or partially by system 10 (and particularly electronic device 50). Thus, in some embodiments, the method 100 may be wholly or partially represented as machine-readable instructions 56 that are stored on memory 54 and executed by processor 52 of electronic device 50. In addition, in describing the method 100, continued reference will be made to system 10 shown in FIG. 1. However, it should be appreciated that method 100 may also be performed with systems that are different from system 10. Thus, the reference to system 10 is merely intended to further illuminate the features of method 100 and should not be interpreted as limiting all possible applications of method 100 in various embodiments.

Initially, method 100 includes receiving an EEG output at block 102. For instance, for the system 10, the output signals from the electrodes 14 may comprise EEG output. The output signals from electrodes 14 are communicated to the electronic device 50 via a suitable connection (e.g., wired connection, wireless connection) and/or network (local area network, wide area network, the Internet, etc.). Upon receipt, the EEG output may be stored in a memory (e.g., memory 54).

Referring still to FIG. 2, after the EEG output is received at block 102, method 100 includes determining a mathematical model of the subject's brain using the EEG output at block 104. For the system 10, the processor 52 may determine the model at block 104 by executing the machine-readable instructions 56 stored on memory 54 and using the EEG output received from the electrodes 14.

The mathematical model (or more simply the “model”) may comprise one or more linear ordinary differential equations (ODEs) (e.g., such as a plurality of ODEs) that approximate the EEG output for the subject. Specifically, one may propose that the human brain is analogous, in some respects, to a mechanical system in that the brain will produce an output (e.g., electrical impulses represented by the EEG output) at characteristic frequencies in response to various inputs, which may comprise chemical or biological stimuli. Thus, like a mechanical system, it is theorized that there are characteristic vibrational frequencies for the human brain, and that it may be possible to derive a mathematical model to predict brain activity. Accordingly, the model determined at block 104 may reveal patterns in the EEG output at the fundamental vibrational frequencies of the subject brain to allow characterization of the subject's brain activity as described in more detail below.

The model determined at block 104 may be determined using Output only (or operational) Modal Analysis (OMA). Specifically, the model is configured to approximate the response of the subject's brain; however, the available data of the subject's brain is the EEG output, which is indicative of the output response to stimuli (e.g., the input to the subject's brain). Thus, a mathematical model determined at block 104 may be determined solely using these output signals via OMA.

In particular, the OMA at block 104 involves deriving the coefficients of the linear ODEs using the EEG output. This process may involve taking a first portion of the EEG output that is associated with a first time period (e.g., T₀-T₁) to determine the coefficients of the linear ODEs of the model. The coefficients may be determined using a regression of analysis of the EEG data over the first time period (e.g., T₀-T₁). Thereafter, the linear ODEs (with the newly derived coefficients) are used to predict the EEG output for a second time period (e.g., T₂-T₃) that is different from the first time period (e.g., T₀-T₁). The error between the predicted EEG output and the actual EEG output for the second time period (e.g., T₂-T₃) may then be used to correct or adjust the coefficients of the linear ODEs. In some embodiments, this process may be repeated a plurality of times by splitting the total EEG output into a plurality of separate time segments, to further refine the coefficients of the linear ODE. As a result, the resulting mathematical model in block 104 is a best-fit linear approximation of the of the brain activity captured via the EEG output received at block 102.

Once the linear ODEs are derived via the process described above, the Eigen Vectors and the Eigen Values of the linear ODEs may be determined. Together, the Eigen Vectors may form a model invariant subspace, and linear combinations of the Eigen Vectors may provide the underlying EEG output used to determine the model as described above. Therefore, the Eigen Vectors may be characterized as a linear decomposition of the EEG output received at block 102. The Eigen Values are proportional (or equal) to the oscillating frequencies of the Eigen Vectors, so that the Eigen Values are also proportional (or equal) to the oscillating frequencies of the EEG output as well. Thus, each Eigen Vector is associated with a corresponding one of the Eigen Values, and each coupled Eigen Vector and Eigen Value may be referred to herein as an “Eigen Mode.” Stated differently, for each Eigen Mode of the model determined at block 104, there is a corresponding Eigen Vector and Eigen Value.

The Eigen Vectors and Eigen Values (i.e., the Eigen Modes) may define the model determined at block 104. The Eigen Vectors and Eigen Values (i.e., the Eigen Modes) may be represented as a matrix. Thus, the model determined at block 104 may comprise a series of linear ODEs, represented as a matrix of Eigen Modes (i.e., combinations of Eigen Vectors and Eigen Values) that provide a prediction of the time/space behavior of the subject's brain (e.g., in terms of the electrical impulses output by the brain over time as detected by an EEG). As will be described below, in some embodiments, this model derived via block 104 may be directly used to characterize the brain activity of the subject (e.g., subject 12) for some circumstances. However, in some embodiments, method 100 may include additional features (e.g., blocks 106, 108, 110) for further refining the model 104 initially derived at block 104 for increasing the accuracy and, in some cases, usefulness of any subsequent characterization of the user brain activity (e.g., at block 112).

In some embodiments, after the model is initially derived at block 104, additional EEG output may be received at block 106. The additional EEG output of block 106 may comprise additional EEG output from the same subject or a different subject that is associated with the EEG output received at block 102. Therefore, in some embodiments, EEG output may be received from a first subject at a first point in time at block 102 and may be used to derive the model at block 104, and then additional EEG output from the first subject at a second point in time that is different (e.g., before, after) from the first point in time may be received at block 106. In addition, in some embodiments, EEG output from a first subject may be received at block 102 and used to determine the model at block 104, and then additional EEG output from a second subject, that is different from the first subject, may be received as the additional EEG output at block 106. With respect to the system 10, the additional EEG output may be received by the electronic device 50 in the same manner as described above for the EEG output of block 102.

At block 108, the method 100 includes updating the model coefficients using the additional EEG output. Updating the model coefficients at block 108 may comprise providing the additional EEG output (which was received at block 106) to the model to determine an error between the predicted brain activity via the model versus the measured activity from the additional EEG data received at block 106. The error may then be used to further refine the coefficients of the model (e.g., the coefficients of the linear ODEs) to further improve the performance of the model. The error between the model and the additional EEG data may be determined using the iterative approach described above for the OMA of block 104. Specifically, a first time portion of the additional EEG output (received at block 106) may be provided to the model, so that the model may make a prediction of a brain activity for a second time portion (where the second time portion is different from the first time portion). The difference between the predicted and actual values of the EEG output for the second time portion may then be taken as the error that is used to then correct the coefficients of the model as previously described. The additional refinement of the model at block 108 may improve the model so that characterizations of brain activity (e.g., at block 112) may be more accurate and/or useful. In some embodiments, when the additional EEG output received at block 106 is associated with a subject (e.g., subject 12) that is different from the subject associated with the EEG output received at block 102, the updates to the coefficients of the model described above for block 108 may act to fit the previously determined model to the brain activity of the individual associated with the EEG output received at block 106. The updating at block 108 may not involve any updates to the underlying Eigen Modes that were previously determined along with the initial model at block 104.

In addition to updating the coefficients of the ODEs in block 108, the method 100 may also include updating the model using an estimation of the input to the subject's brain at block 110. In particular, block 110 may comprise updating the Eigen Vectors and/or the Eigen Values of the Eigen Modes of the model determined at block 104 by estimating an input to the subject's brain based on the additional EEG output received at block 106 and then providing the estimated input to the model to compute an error for the Eigen Vectors and/or the Eigen Values. As previously described, the input to the subject's brain may comprise chemical and/or biological stimuli and is unknown. However, a mathematical estimation of the input to the subject's brain may be derived based on the output (the EEG output), and this estimated input may then be used to further refine the Eigen Modes of the model initially determined at block 104 to further fit the model to the brain activity observed in the updated EEG output received at block 106.

In some embodiments, the mathematical representation of the input of the subject's brain (in the same coordinate space as the EEG output) is estimated at block 110 using an adaptive parameter estimator that captures the time varying dynamics in the additional EEG output received at block 106. The adaptive parameter estimator comprises an ODE (or a set of ODEs) that models the mathematical representation of the unknown input to the subject's brain as a linear combination of basis functions. The basis functions comprise a selected set of functions that best approximate the inputs and outputs from the system being analyzed (e.g., in this case, the human brain). The output from the brain (e.g., in terms of an EEG output) is spectral, meaning it is in the form of sines and cosines. Accordingly, it is assumed that the input to the brain, if placed in the same coordinate space as the EEG output, would also be spectral. Accordingly, estimating the input at block 110 may comprise selecting the suitable sines and cosines (or combinations thereof) as the basis functions. In some embodiments, the unknown input is estimated at block 110 by isolating the portions of the additional EEG output that may not be represented by the Eigen Modes determined via OMA in block 104.

More particularly, in some embodiments updating the model at block 110 may comprise generating an estimate of the unknown input to the subject's brain by identifying the weighting coefficients for the selected basis functions (e.g., spectral functions as described above) which minimize the difference between the EEG output predicted by the model and the observed EEG output from the additional EEG output received at block 106. EEG output (e.g., the EEG output received at blocks 102, 106) has zero mean and exhibits periodic band-limited behavior. Therefore in some embodiments a Fourier series of the waveforms which make up the unknown input are utilized to estimate the input at block 110. Thus, the updating the model at block 110 comprises selecting a best fit set of sine and cosine waveforms which the ODEs of the model determined at block 104 are restricted from generating due to the formulation of the model.

In addition, updating the model at block 110 also comprises updating the Eigen Modes themselves to adapt the model to unseen EEG output. Specifically, an adaptive gain law, which is driven by the model error and observed EEG output, may be use to update the Eigen Modes to correct the model. The unknown input and Eigen Modes update are performed simultaneously in the estimator, resulting in a greatly improved model.

Referring still to FIG. 2, after the model is updated via blocks 108 and 110, method 100 may proceed to characterize the brain activity of the subject using the model at block 112. As previously described, in some embodiments, method 100 may progress to block 112 after block 104, and without updating the model per blocks 106, 108, 110. In some embodiments, method 100 may progress to block 112 after updating the model per blocks 106, 108, 110 (including updating the model per blocks 106 and 108, per blocks 106 and 110, or per blocks 106, 108, and 110).

In some embodiments, characterizing the brain activity of the subject at block 112 comprises identifying the identity of the particular subject that contributed the EEG output (e.g., the EEG output received at block 102 or the additional EEG output received at block 106). For instance, once the model is determined at block 104 and potentially refined with the additional EEG output received at block 106 via one or both of blocks 108, 110, the Eigen Modes (including the corresponding Eigen Vectors and Eigen Values) of the model may be used to identify the specific individual who contributed the EEG output. Specifically, it is postulated herein that some portion of the Eigen Modes of the model determined at block 104 may be common to all (or substantially all) individuals. In other words, there are some portion of brain output signals (as detected via the EEG output) that are common for all (or substantially all) human brains. However, some portion of the Eigen Modes of the model determined at block 104 may be specific to each individual. These characteristic features of brain activity may function like a fingerprint that can then be used to identify a specific individual who is associated with a given EEG output (e.g., subject 12 in FIG. 1). Thus, in some embodiments, block 112 may comprise comparing the Eigen Modes (or some portion thereof) of the model determined at block 104 (and potentially refined via blocks 106, 108, 110) to a database of Eigen Mode profiles for a plurality of individuals. In some embodiments, Eigen Modes that are common to all (or substantially all) individuals may not be used for the comparison. The comparison may yield a profile which presents the closest match to the Eigen Modes of the model. The matched profile is associated with a specific individual who is then selected as the identified individual associated with the EEG output. In some embodiments, a minimum correspondence (e.g., a maximum error value) may be defined between the Eigen Modes of the model and the Eigen Modes of the profiles of the database to avoid identifying an individual when a sufficiently close match is not determined from the comparison.

The individual identified via block 112 may be the subject who contributed the EEG output at block 102 or the subject who contributed the additional EEG output at block 106. Specifically, as previously described, in some embodiments, the method 100 may progress to block 112 after block 104 and without performing blocks 106, 108, 110. Thus, in this circumstance, the individual identified at block 112 may be the individual who contributed the EEG output from block 102, which was used to determine the model at block 104. Conversely, in some embodiments, where the model is updated per blocks 106, 108, 110 (or perhaps only blocks 106 and 110 or only blocks 106 and 108), the individual identified in block 112 may comprise the individual who contributed the additional EEG output at block 106 (which may be the same or a different individual who contributed the EEG output at block 102 as previously described).

In some embodiments, the comparison at block 112 may comprise comparing the Eigen Vectors of the Eigen Modes to the Eigen Vectors of the plurality of profiles. In other words, the comparison may not include a comparison of the Eigen Values of the model with those of the plurality of profiles. In some embodiments, the comparison at block 112 may be carried out via a machine-learning model that receives the Eigen Modes (or Eigen Vectors) of the model as an input and that classifies the Eigen Modes (or Eigen Vectors) as being associated with a particular one of the plurality of profiles in the database. For instance, in some embodiments, the machine-learning model may comprise a regression model (e.g., a linear regression model), a neural network, or any other suitable machine-learning model for classification of data.

In some embodiments, characterizing the brain activity of the subject at block 112 comprises identifying a cognitive state of the subject that provided the EEG output (e.g., the EEG output of block 102 or the additional EEG output of block 106). In particular, identifying the cognitive state of the subject may comprise selecting a cognitive state from a plurality of defined cognitive states using the model. In some embodiments, the cognitive state that is identified at block 112 may be defined via any suitable theory or scale. In some embodiments, the cognitive state is defined using Russell's Valence-Arousal Scale, such that the cognitive state of the subject may be defined as being one of high valence or low valence and one of high arousal or low arousal.

High valence may be associated with positive feelings or reception by the individual, whereas low valence may be associated with negative feelings or reception by the individual. In addition, the arousal level may correspond the level of enthusiasm or excitement of thought the individual has toward the current subject matter, person, or activity. Thus, a high arousal may correspond with a high level of enthusiasm or excitement and a lower arousal may correspond with a low level of enthusiasm or excitement. Taken together, the valence and arousal levels may allow one to characterize the general cognitive state of the individual with respect to some subject matter (e.g., an image, video, activity, object, statement, etc.). So, for instance, a subject who is greatly offended by a particular subject matter may have a high arousal but a low valence, because the subject may have a great level of interest or excitement for the subject matter but a negative reaction to the subject matter overall. Conversely, a subject who is mildly amused by a particular subject matter (e.g., such as a reaction to what may be perceived as an exhibition of childish humor) may have a low excitement but a high valence, because the individual may have a low level of interest or excitement for the subject matter but a generally positive reaction to the subject matter overall.

Thus, in some embodiments, when block 112 comprises identifying the cognitive state of the subject, block 102 may comprise receiving a first EEG output that is associated with high valence, a second EEG output that is associated with a low valence, a third EEG output that is associated with high arousal, and a fourth EEG output that is associated with low arousal. In other words, in these circumstances, the EEG output received at block 102 (including the first, second, third, and fourth EEG outputs) may comprise so-called “labeled” or known data for each of the potential valence and arousal states or levels (e.g., high valence, low valence, high arousal, low arousal). Thereafter, the model determined at block 104 may comprise four separate models, a first model associated with high valence that is determined using the first EEG output, a second model associated with low valence that is determined using the second EEG output, a third model associated with high arousal that is determined using the third EEG output, and a fourth model associated with low arousal that is determined using the fourth EEG output. Each of the first, second, third, and fourth models may comprise ODEs that define a plurality of corresponding Eigen Modes (including associated Eigen Vectors and Eigen Values) as previously described. However, each of the ODEs and Eigen Modes associated with the first, second, third, and fourth models may be difference based on the valence/arousal state the models are associated with as described above.

Next, the additional EEG output may be received at block 106 as previously described. In this case, the additional EEG output comprises EEG output associated with a subject for whom the cognitive state is to be characterized via block 112. As previously described, the additional EEG output received at block 106 may be from a different subject than the subject or subjects that provided the EEG output at block 102 that is associated with the first, second, third, and fourth models from block 104. In some embodiments, the additional EEG output received at block 106 may be associated with the same subject who provided the EEG output at block 102 that is associated with the first, second, third, and fourth models from block 104.

The additional EEG output may then be used to update the first, second, third, and fourth models via blocks 108 and 110 (or perhaps only via block 108 or only via block 110 in some embodiments), in the manner previously described above. During this process, the first, second, third, and fourth models may each eventually be updated such that they all generally coalesce to the same model (or substantially the same model). However, the relative errors for updating the models during this process may be different. As a result, in block 112, the errors between the first, second, third, and fourth models that were computed during the updating process of blocks 108 and/or 110 may indicate which of the valence and arousal states is associated with the additional EEG output received at block 106.

Specifically, consideration will first be provided to the first and second models, which are associated with high and low valence as previously described. If the error associated with the first model is greater than the error associated with the second model when updating the models per blocks 108 and/or 110, then a determination at block 112 may be made that the additional EEG output received at block 106 may be more closely associated with the second model, and therefore a low valence (or low positivity). Conversely, if the error associated with the second model is greater than the error associated with the first model when updating the models per blocks 108 and/or 110, then a determination at block 112 may be made that the additional EEG output received at block 106 may be more closely associated with the first model, and therefore a high valence (or high positivity).

Consideration will now be provided to the third and fourth models, which are associated with a high and low arousal as previously described. If the error associated with the third model is greater than the error associated with the fourth model when updating the models per blocks 108 and/or 110, then a determination at block 112 may be made that the additional EEG output received at block 106 may be more closely associated with the fourth model, and therefore a low arousal. Conversely, if the error associated with the fourth model is greater than the error associated with the third model when updating the models per blocks 108 and/or 110, then a determination at block 112 may be made that the additional EEG output received at block 106 may be more closely associated with the third model, and therefore a high arousal.

In this manner, by comparing the error rates between the first, second, third, and fourth models during the updating (e.g., at blocks 108 and/or 110) per the additional EEG output received at block 106, a determination may be made at block 112 as to whether the individual who contributed the additional EEG output at block 106 had a high or low valence and a high or low arousal during the time period associated with the EEG output. This information may be useful in situation where one wishes to know a subject's first impression of a product, advertisement, film, etc. without relying on the verbal feedback from the individual. Thus, in some circumstances, an individual may be coupled to electrodes (e.g., electrodes 14) while viewing and/or interacting with a particular object or subject matter, and the determination of the individual's cognitive state may be made as described above for block 112 so that useful conclusions and feedback of the individual's reactions to the object or subject matter without directly asking for verbal feedback from the individual.

The embodiments disclosed herein include methods and associated systems for characterizing the brain activity of a subject via output obtained from an EEG. In some embodiments, the systems and methods disclosed herein may generate a mathematical model(s) for approximating the function of the subject's brain. These models may allow isolation of the underlying linear and non-linear patterns associated with the EEG output signals, which may then be used to make useful characterizations of the brain activity of the subject. Thus, through use of the systems and methods disclosed herein, further useful insights may be obtained from an EEG (either along or along with other imaging or analysis techniques such as fMRI) which may facilitate further understanding of human thought and brain function.

The following discussion is directed to various exemplary embodiments. However, one of ordinary skill in the art will understand that the examples disclosed herein have broad application, and that the discussion of any embodiment is meant only to be exemplary of that embodiment, and not intended to suggest that the scope of the disclosure, including the claims, is limited to that embodiment.

The drawing figures are not necessarily to scale. Certain features and components herein may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . . ” Also, the term “couple” or “couples” is intended to mean either an indirect or direct connection. Thus, if a first device couples to a second device, that connection may be through a direct connection of the two devices, or through an indirect connection that is established via other devices, components, nodes, and connections. In addition, as used herein, the terms “axial” and “axially” generally mean along or parallel to a given axis (e.g., central axis of a body or a port), while the terms “radial” and “radially” generally mean perpendicular to the given axis. For instance, an axial distance refers to a distance measured along or parallel to the axis, and a radial distance means a distance measured perpendicular to the axis. Further, when used herein (including in the claims), the words “about,” “generally,” “substantially,” “approximately,” and the like mean within a range of plus or minus 10%.

While exemplary embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

1. A method of characterizing brain activity, the method comprising:
receiving an electroencephalogram (EEG) output;
determining a mathematical model of a brain using the EEG output, wherein the mathematical model comprises a plurality of ordinary differential equations (ODEs) that are determined based on the EEG output; and
characterizing brain activity of a subject using the mathematical model.

2. The method of claim 1, wherein determining the mathematical model of the subject's brain comprises applying output-only modal analysis (OMA) to the EEG output.

3. The method of claim 2, wherein characterizing the brain activity of the subject comprises:
comparing a plurality of Eigen Modes of the model to a plurality of Eigen Mode profiles; and
identifying an identity of the subject based on the comparison.

4. The method of claim 2, wherein characterizing the brain activity of the subject comprises selecting a cognitive state from a plurality of cognitive states using the mathematical model.

5. The method of claim 4, wherein determining a mathematical model of the brain using the EEG output comprises determining a plurality of mathematical models, wherein each of the plurality of mathematical models is associated with a corresponding one of the plurality of cognitive states, and
wherein selecting the cognitive state from the plurality of cognitive states comprises:
applying additional EEG output from the subject to the plurality of mathematical models;
comparing an error from each of the plurality of mathematical models that results from applying the additional EEG output to the plurality of models; and
selecting the cognitive state based on the comparison.

6. The method of claim 1, comprising:
receiving additional EEG output from the subject; and
updating the model based on the additional EEG output.

7. The method of claim 6, wherein updating the model based on the additional EEG output comprises:
estimating an input for the model to generate the additional EEG output; and
updating a plurality of Eigen Modes for the model based on the input.

8. The method of claim 7, wherein estimating the input for the model comprises computing the input as a linear combination of spectral basis functions.

9. A non-transitory, machine-readable medium, storing instructions, which, when executed by a processor of an electronic device, cause the processor to:
receive an electroencephalogram (EEG) output;
determine a mathematical model of a brain using the EEG output, wherein the mathematical model comprises a plurality of ordinary differential equations (ODEs) that are determined based on the EEG output; and
characterize brain activity of a subject using the mathematical model.

10. The non-transitory, machine-readable medium of claim 9, wherein the instructions, when executed by the processor, cause the processor to apply output-only modal analysis (OMA) to determine the mathematical model of the subject's brain based on the EEG output.

11. The non-transitory, machine-readable medium of claim 10, wherein the instructions, when executed by the processor, cause the processor to characterize the brain activity of the subject by:
comparing a plurality of Eigen Modes of the model to a plurality of Eigen Mode profiles; and
identifying an identity of the subject based on the comparison.

12. The non-transitory, machine-readable medium of claim 10, wherein the instructions, when executed by the processor, cause the processor to characterize the brain activity of the subject by selecting a cognitive state from a plurality of cognitive states using the mathematical model.

13. The non-transitory, machine-readable medium of claim 12, wherein the instructions, when executed by the processor, cause the processor to determine a mathematical model of the brain using the EEG output by determining a plurality of mathematical models, wherein each of the plurality of mathematical models is associated with a corresponding one of the plurality of cognitive states, and
wherein the instructions, when executed by the processor, cause the processor to select the cognitive state from the plurality of cognitive states by:
applying additional EEG output from the subject to the plurality of mathematical models;
comparing an error from each of the plurality of mathematical models that results from applying the additional EEG output to the plurality of models; and
selecting the cognitive state based on the comparison.

14. The non-transitory, machine-readable medium of claim 9, wherein the instructions, when executed by the processor, cause the processor to:
receive additional EEG output from the subject; and
update the model based on the additional EEG output.

15. The non-transitory, machine-readable medium of claim 14, wherein the instructions, when executed by the processor, cause the processor to update the model based on the additional EEG output by:
estimating an input for the model to generate the additional EEG output; and
updating a plurality of Eigen Modes for the model based on the input.

16. The non-transitory, machine-readable medium of claim 15, wherein the instructions, when executed by the processor, cause the processor to estimate the input for the model by computing the input as a linear combination of spectral basis functions.

17. A system, comprising:
a plurality of electrodes that are configured to detect electrical impulses within a brain of a subject; and
an electronic device coupled to the plurality of electrodes, wherein the electronic device is configured to:
receive an electroencephalogram (EEG) output;
determine a mathematical model of a brain using the EEG output, wherein the mathematical model comprises a plurality of ordinary differential equations (ODEs) that are determined based on the EEG output;
receive additional EEG output from the subject from the plurality of electrodes;
update the mathematical model using the additional EEG output; and
characterize brain activity of the subject using the mathematical model.

18. The system of claim 17, wherein the electronic device is configured to update the mathematical model of the brain by:
estimating an input for the model to generate the additional EEG output; and
updating a plurality of Eigen Modes for the model based on the input.

19. The system of claim 18, wherein the electronic device is configured to estimate the input for the model by computing the input as a linear combination of spectral basis functions.

20. The system of claim 19, wherein the electronic device is configured to update the plurality of Eigen Modes for the model by:
applying the input to the model to generate a predicted EEG output;
determining an error between the predicted EEG output and the additional EEG output; and
updating the Eigen Modes of the mathematical model based on the error.
